(19) United States
(12) Patent Application Publication
(10) Pub. No.: US 2008/0193537 A1
(43) Pub. Date: Aug. 14, 2008

(54) Morphine Sulfate Formulations

(75) Inventor: Alfred C. Liang, Edison, NJ (US)

Correspondence Address: CANTOR COLBURN, LLP, 20 Church Street, 22nd Floor, Hartford, CT, 06103, US

(73) Assignee: ALPHARMA, INC., Bridgewater, NJ (US)

(21) Appl. No.: 11/913,699

(22) PCT Filed: May 12, 2006
(86) PCT No.: PCT/US06/18918

§ 371 Date: Apr. 14, 2008

Related U.S. Application Data
(60) Provisional application No. 60/680,554, filed on May 13, 2005.
(60) Provisional application No. 60/682,977, filed on May 20, 2005.

(51) Int. Cl. A61K 9/00; A61K 31/485

(52) U.S. Cl. 424/487; 424/484; 424/486; 514/282

(57) ABSTRACT

A dosage form comprising a plurality of pellets, the pellets comprising a core element comprising morphine sulfate, a filler and a binder, wherein the morphine sulfate, calculated as the anhydrous form, comprises about 50 wt % to about 85 wt % of the total weight of the core element; and a controlled-release coating disposed on at least a portion of the core element, the coating comprising an insoluble matrix polymer which is insoluble at pH 1 to 7.5; an enteric polymer which is insoluble at pH 1 to 4 and soluble polymer which is soluble at a pH of 1 to 4, wherein the ratio of the acid soluble polymer to the enteric polymer is 1.45:1 to 2.5:1 on a weight basis, wherein the Cmax of the dosage form differs by less than 20% when administered to a mammalian subject in the fed state compared to the fasted state. Also included are methods of increasing patient compliance by administering the disclosed dosage form to a mammalian subject.

### BACKGROUND

Morphine sulfate [7,8-d]dehydro-4,5-(alpha)-epoxy-17-methyl-morphinan-3,6(alpha) (salt) pentahydrate] is an opioid compound with specific affinity for the receptors μ, δ and κ. The principal actions of therapeutic value are analgesia and sedation. The precise mechanism of the analgesic action is unknown. Specific opioid receptors have been located in the brain and the spinal cord and are likely to play a role in the expression of analgesic effects.

Morphine is regarded as the opioid drug of choice in the treatment of cancer pain, for example. Side effects of morphine treatment include, for example, nausea and vomiting, constipation, sedation, confusion and loss of appetite. It has been suggested that the use of modified release morphine formulations, apart from their convenience and their ability to provide continuous analgesia, may also result in a lower incidence and severity of morphine-related side effects. Sustained-release morphine dosage forms are described in U.S. Pat. Nos. 5,202,128 and 5,378,474.

Kadian® is a morphine sustained-release dosage form for once or twice per day dosing. Kadian® is currently available in 20, 30, 50, 60 and 100 mg capsules comprising sustained-release pellets of morphine sulfate.

The present invention addresses the need for improved morphine dosage forms, particularly high dose forms.

### SUMMARY

In one embodiment, a dosage form comprises a plurality of pellets, the pellets comprising:

a core element comprising morphine sulfate, a filler and a binder, wherein the morphine sulfate, calculated as the anhydrous form, comprises about 50 wt % to about 85 wt % of the total weight of the core element; and

a controlled-release coating disposed on at least a portion of the core element, the coating comprising an insoluble matrix polymer which is insoluble at pH 1 to 7.5; an enteric polymer which is insoluble at pH 1 to 4 and soluble at pH 6 to 7.5; and an acid soluble polymer which is soluble at a pH of 1 to 4, wherein the ratio of the acid soluble polymer to the enteric polymer is 1.45:1 to 2.5:1 on a weight basis,

wherein the Cₘₐₓ of the dosage form differs by less than 20% when administered to a mammalian subject in the fed state compared to the fasted state.

A method of increasing patient compliance comprises administering to a patient in need thereof the above dosage form.

### BRIEF DESCRIPTION OF THE DRAWINGS

Referring now to the drawings wherein like elements are numbered alike in several FIGURES:

FIG. 1 shows the release profile of Comparative Example 1 at pH 7.5

FIG. 2 shows the pH 7.5 release profiles of Comparative Example 2 at different coating weights.

FIG. 3 shows the release profiles of a Comparative Example 2 below pH 6.

FIG. 4 shows the release profiles for the pellet formulations of Examples 1-3 at pH 4.5.

FIG. 5 shows the release profiles for the formulation of Example 4 at various pHs.

FIG. 6 shows the release profiles for the formulation of Example 5 at various pHs.

The above-described and other features will be appreciated and understood by those skilled in the art from the following detailed description, drawings, and appended claims.

### DETAILED DESCRIPTION

### Chemical Description and Terminology

The use of the terms “a” and “an” and “the” and similar referents (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein, the terms wt %, weight percent, percent by weight, etc. are equivalent and interchangeable.

The term “active agent” is meant to include solvates (including hydrates) of the free compound or salt, crystalline and non-crystalline forms, as well as various polymorphs. Unless otherwise specified, the term “active agent” is used herein to indicate morphine or a pharmaceutically acceptable salt thereof. For example, an active agent can include all optical isomers of morphine and all pharmaceutically acceptable salts thereof either alone or in combination.

By “oral dosage form” is meant to include a unit dosage form prescribed or intended for oral administration. An oral dosage form may or may not comprise a plurality of subunits such as, for example, microcapsules or microtablets, packaged for administration in a single dose.

By “subunit” is meant to include a composition, mixture, particle, etc., that can provide an oral dosage form alone or when combined with other subunits. By “part of the same subunit” is meant to refer to a subunit comprising certain ingredients.

Dissolution profile as used herein, means a plot of the cumulative amount of active ingredient released as a function of time. The dissolution profile can be measured utilizing the Drug Release Test <724>, which incorporates standard test USP 26 (Test <711>). A profile is characterized by the test conditions selected. Thus the dissolution profile can be generated at a preselected apparatus type, shaft speed, temperature, volume, and pH of the dissolution media.

Release forms may also be characterized by their pharmacokinetic parameters. “Pharmacokinetic parameters” are parameters which describe the in vivo characteristics of the active agent over time, including for example the in vivo dissolution characteristics and plasma concentration of the active agent. By “Cₘₐₓ” is meant the measured concentration of the active agent in the plasma at the point of maximum concentration. By “C₂₄” is meant the concentration of the active agent in the plasma at about 24 hours. The term “Tₘₐₓ” refers to the time at which the concentration of the active agent in the plasma is the highest. “AUC” is the area under the curve of a graph of the concentration of the active agent (typically plasma concentration) vs. time, measured from one time to another.

By “instant-release” is meant a dosage form designed to ensure rapid dissolution of the active agent by modifying the normal crystal form of the active agent to obtain a more rapid dissolution. By “immediate-release”, it is meant a conventional or non-modified release in which greater than or equal to about 75% of the active agent is released within two hours of administration, preferably within one hour of administration.

By “controlled-release” it is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate release and controlled release characteristics, for example, a combination of immediate release pellets and controlled release pellets. The immediate release portion of the dosage form may be referred to as a loading dose.

Certain formulations described herein may be “coated”. The coating can be a suitable coating, such as, a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

As used herein, a fasted patient is a patient who fasts for at least 10 hours before the administration of a morphine sulfate dosage form and who continues to fast for at least 4 hours after the administration of the dosage form. The dosage form is administered with water during the fasted period, and water is permitted after 2 hours.

As used herein, a fed patient is a patient who fasts for at least 10 hours overnight and then consumes an entire test meal within 30 minutes of first ingestion of the morphine sulfate dosage form. The dosage form is administered with water within 5 minutes after completion of the meal. No food is then allowed for at least 4 hours post-dose. Water can be allowed after 1 hours. A high fat test meal provides approximately 1000 calories to the patient of which approximately 50% of the caloric content is derived from fat content of the meal. A representative high fat high calorie test meal comprises 2 eggs fried in butter, 2 strips of bacon, 2 slices of toast with butter, 4 ounces of hash brown potatoes, and 8 ounces of whole milk to provide 150 protein calories, 250 carbohydrate calories, and 500 to 600 fat calories.

### Dosage Forms

A pellet composition comprises a core element comprising morphine compound (e.g., morphine sulfate) and a controlled-release coating disposed on at least a portion of the core element, wherein the coating is partially soluble at a highly acidic pH to provide a slow rate of release of the morphine sulfate. The morphine sulfate may be available for absorption at a relatively constant faster rate in the intestine over an extended period of time. A plurality of pellets may be combined to form a morphine sulfate dosage form. The disclosed high dose form comprises a core element comprising morphine sulfate, a filler and a binder, wherein the morphine sulfate comprises greater than or equal to about 50 wt % of the total weight of the core element. The core element is coated with a controlled-release coating. The dosage form, in use, exhibits less fluctuations in plasma concentrations in morphine sulfate at steady state over a 24 hour period, relative to the active ingredient in an uncoated form and/or exhibits less diurnal variation in plasma concentration of active ingredient relative to known capsules or tablets containing the at least one active ingredient in a sustained release form.

Kadian® is an FDA approved sustained-release morphine dosage form with approved doses of 20, 30, 50, 60, and 100 mg capsules containing polymer coated sustained release pellets of morphine sulfate. Virtually all of the morphine is converted to glucuronide metabolites, including morphine-3-glucuronide and morphine-6-glucuronide that occur in the highest concentrations in the plasma following oral administration of morphine.

The absorption and bioavailability of a formulation comprising an active agent, including sustained release formulations, can be affected by numerous factors when dosed orally. Such factors include, but are not limited to, the presence of food in the gastrointestinal (GI) tract. The presence of food in the GI tract may cause the gastric residence time of an active agent to be significantly longer than if administered in the fasted state. If the bioavailability of an active agent is affected beyond a certain point due to the presence of food in the GI tract, the active agent is said to exhibit a “food effect”. Concurrent administration of food slows the rate of absorption of morphine from Kadian®, however the extent of absorption does not appear to be affected.

The term “no fed effect” or “no food effect” as used herein means that there is less than a 20% difference between the pharmacokinetic parameters (determined from blood levels of active agent or its metabolites) with respect to the values for Cₘₐₓ and AUC obtained when patients are dosed with the formulation on an empty stomach as compared to when the formulation is administered to patients who have ingested a high-fat meal. A high fat meal is as defined by the U.S. Food and Drug Administration or corresponding foreign regulatory body (i.e., the “fed state”). A food effect is considered to exist where these differences when dosed in the fed versus the fasted state are greater than 20%. The measured blood levels can be those for morphine or for one of its metabolites such as morphine-3-glucuronide.

Disclosed herein is a morphine dosage form comprising a core element comprising morphine sulfate, wherein at least a portion of the core element is coated with a controlled-release coating, wherein the dosage form has no food effect. The lack of a food effect is significant as it eliminates the need for a patient to ensure that they are taking a dose with or without food. Therefore, the disclosed dosage form will result in increased patient compliance. With poor patient compliance, an increase in pain for which the morphine sulfate is being prescribed can result. Thus, a method of increasing patient compliance comprises proving to a patient in need thereof the disclosed morphine dosage form.

The invention encompasses morphine sulfate dosage forms wherein the pharmacokinetic profile of the morphine sulfate is not substantially affected by the fed or fasted state of a subject ingesting the dosage form. Specifically, there is no substantial difference in the rate or quantity of drug absorption when the morphine sulfate dosage form is administered in the fed versus the fasted state. Thus, the morphine sulfate dosage forms substantially eliminate the effect of food on the pharmacokinetics of the morphine sulfate. The invention also encompasses a morphine sulfate dosage form in which administration of the composition to a subject in a fasted state is bioequivalent to administration of the composition to a subject in a fed state.

The invention thus encompasses morphine sulfate dosage forms in which administration of the dosage form to a subject in a fasted state is bioequivalent to administration of the dosage form to a mammalian subject in a fed state. In one embodiment, the mammalian subject is a human subject. The difference in Cₘₐₓ, AUC_{0-∞}, Tₘₐₓ, or a combination comprising one or more of the foregoing pharmacokinetic parameters for the morphine sulfate dosage form composition, when administered in the fed versus the fasted state, is less than about 20%, less than about 15%, less than about 10%, or less than about 5%. In one embodiment, the difference in Cₘₐₓ for the morphine sulfate dosage form, when administered in the fed versus the fasted state, is less than about 20%, less than about 15%, less than about 10%, or less than about 5%. In another embodiment, the difference in Cₘₐₓ and AUC_{0-∞} for the morphine sulfate dosage form, when administered in the fed versus the fasted state, is less than about 20%, less than about 15%, less than about 10%, or less than about 5%.

The controlled-release morphine formulation is based on pellets comprising a core element comprising morphine sulfate, a filler, and a binder. The morphine sulfate may be present in an anhydrous or hydrous form. The morphine sulfate is present in amounts, calculated as the anhydrous form, of about 50 wt % to about 85 wt % of the total weight of the core element, specifically 60 wt % to about 80 wt % of the core element. In one embodiment, the morphine sulfate is in the form of morphine sulfate pentahydrate.

Suitable binders include, for example, polyvinyl pyrrolidone, hydroxypropyl cellulose, hydroxypropyl methylcellulose, methylcellulose, hydroxyethyl cellulose, sugars, and combinations comprising one or more of the foregoing binders. The binder may be provided in the form of a granulating solution optionally including an aqueous or organic solvent such as, for example, methanol, ethanol, and mixtures thereof. The binder comprises about 1 wt % to about 10% of the total weight of the core element.

Suitable fillers include, for example, silicon dioxide, talc, titanium dioxide, alumina, starch, kaolin, polacrilin potassium, powdered cellulose, and microcrystalline cellulose, and combinations comprising one or more of the foregoing fillers. Soluble fillers include, for example, mannitol, sucrose, lactose, dextrose, sodium chloride, sorbitol, and combinations comprising one or more of the foregoing fillers. In certain embodiments, the filler acts as an osmotic agent in the core. The core element may comprise about 4 wt % to about 45 wt % of the filler, specifically about 10 wt % to about 30 wt %.

In one embodiment, the filler is in the form of an inert core onto which the morphine sulfate and the binder are coated. Suitable inert cores include for example, sugar spheres, particulate microcrystalline cellulose, silicon dioxide spheres, wax beads such as prilled waxes, and combinations comprising one or more of the foregoing inert cores. The size and amount of the inert core may vary substantially from about 300 um to about 1200 um depending upon the amount of active ingredient to be included. Accordingly, the inert core may vary from about 30 wt % to about 40 wt %, specifically about 25 wt % to about 35 wt % of the total weight of the core element. In one embodiment, the inert core comprises nonpareil sugar seeds having an average size of about 18 to about 20 mesh (850 to 1000 micrometers). A composition comprising morphine sulfate is disposed on at least a portion the inert cores in an amount sufficient to provide a dosage form comprising about 50 to about 500 mg of morphine sulfate (e.g., 50 mg, 100 mg, 200 mg and 500 mg). In one embodiment, the morphine sulfate is disposed substantially uniformly on the inert core.

The core element may further include other carriers or excipients, such as, for example, stabilizing agents, colorants, and combinations comprising one or more of the foregoing additives.

In one embodiment, the core element is formed by coating an inert core with the morphine sulfate and the binder. The binder and the morphine sulfate may be provided in the form of a solution or slurry. In this form, the inert core may be sprayed with the solution or slurry. Spraying may be conducted in suitable coating equipment such as, for example, a fluidized bed chamber, such as a rotary fluid bed machine.

In another embodiment, when the binder is in the form of a granulation solution, the binder and the morphine sulfate may be coated onto the inert core in a spheronization process. The spheronization process includes contacting the inert core with the morphine sulfate and simultaneously adding the granulating solution thereto. The spheronization process may be conducted in a spheronizing machine.

In a further embodiment, the core element may be formed by subjecting the morphine sulfate, the binder, the filler and a solvent to an extrusion followed by marumerisation to form a core element.

The core elements (e.g., sugar spheres comprising a morphine compound or extruded pellets) are then coated with a controlled-release coating that provides for the controlled release of morphine. The coating comprises an insoluble matrix polymer which is substantially insoluble independent of pH (e.g., insoluble at pH 1 to 7.5); an enteric polymer which is substantially insoluble at acidic pH but at least partially soluble at a less acidic to basic pH (e.g., insoluble at pH 1 to 4 and soluble at pH 6 to 7.5); and an acid soluble polymer which is at least partially soluble at acidic pH (e.g., soluble at pH 1 to 4); wherein the ratio of the acid soluble polymer to the enteric polymer is 1.45:1 to 2.5:1 on a weight basis, specifically 1.5:1 to 2:1. In one embodiment, the enteric polymer is readily soluble at a less acidic to basic pH. In another embodiment, the at least partially soluble component is a readily water-soluble component.

The insoluble matrix polymer may be a suitable pharmaceutically acceptable polymer substantially insoluble independent of pH. Suitable insoluble matrix polymers include, for example, ethylcellulose, acrylic and/or methacrylic ester polymers, and combinations comprising one or more of the foregoing polymers. Polymers or copolymers of acrylates or methacrylates having a low quaternary ammonium content may be employed. In one embodiment, the insoluble matrix polymer comprises ethylcellulose.

The insoluble matrix polymer may be present in the coating in an amount of about 1 wt % to about 85 wt %, specifically about 35 wt % to about 65 wt %, based on the total weight of the coating excluding the weight of filler and plasticizer.

Suitable enteric polymers include, for example, cellulose acetate phthalate, hydroxypropyl methylcellulose phthalate (HPMCP), polyvinyl acetate phthalate, methacrylic acid copolymer, hydroxypropyl methylcellulose acetate succinate, shellac, cellulose acetate trimellitate, and combinations comprising one or more of the foregoing enteric polymers. The methacrylic acid:acrylic acid ethylester 1:1 copolymer sold under the trade designation “Eudragit L100-55” has been found to be suitable.

The enteric polymer may be present in the coating in an amount of about 1 wt % to about 24 wt %, specifically about 10 wt % to about 20 wt %, based on the total weight of the coating excluding the weight of filler and plasticizer.

Suitable acid-soluble polymers include, for example, polyvinyl pyrrolidone, hydroxypropyl cellulose, hydroxypropyl methylcellulose, polyethylene glycol having a molecular weight of 1700 to 20,000, polyvinyl alcohol and monomers therefor such as sugars, salts, or organic acids, and combinations comprising one or more of the foregoing polymers. In one embodiment, the acid soluble polymer is polyethylene glycol having a molecular weight of 1700 to 20,000

The acid-soluble polymer may be present in the coating in amounts of about 25 wt % to about 60 wt %, specifically about 25 wt % to about 50 wt %, based on the total weight of the coating excluding the weight of filler and plasticizer.

The coating may further optionally include at least one plasticizer; and optionally at least one filler. Suitable plasticizers include, for example, diethyl phthalate, triethyl citrate, triethyl acetyl citrate, triacetin, tributyl citrate, polyethylene glycol having a molecular weight of about 200 to less than about 1700, glycerol, and combinations comprising one or more of the foregoing plasticizers. The plasticizer comprises 0 wt % to about 50 wt % of the total weight of the coating. Suitable fillers include, for example, silicon dioxide, titanium dioxide, talc, alumina, starch, kaolin, polacrilin potassium, powdered cellulose, and microcrystalline cellulose and mixtures thereof. The filler comprises 0 wt % to about 75 wt % of the total weight of the coating.

The coating may be disposed on the inert core in the form of a coating composition such as a solution, dispersion or suspension. When the coating composition is in the form of a solution, the solvent may be present in amounts of about 25 wt % to about 97 wt %, specifically about 85 wt % to about 97 wt %, based on the total weight of the coating composition. The solvent may comprise, for example, water, methanol, ethanol, methylene chloride, and combinations comprising one or more of the foregoing solvents. In the form of a dispersion or suspension, the diluting medium may be present in amounts of about 25 wt % to about 97 wt %, specifically about 75 wt % to about 97 wt %, based on the total weight of the coating composition. The diluting medium may comprise about 80% to about 100% v/v of water.

Spray coating of core elements may be performed with bottom, top or tangentially located spray nozzles. A bottom spray nozzle may reside proximate to the base of the fluidized bed facing upwards while a top spraying nozzle is located above the contents of the bed and facing downwards. The spray nozzle may reside in the mid-section of the fluidized bed and be oriented such as to spray tangentially to the rotating core elements.

Once applied and dried, the controlled-release coating may comprise about 8 wt % to about 17 wt % of the total weight of the coated cores, or about 10 wt % to about 13 wt % of the total weight of the coated cores.

The controlled-release coated core elements may be placed in a gelatin capsule or they may be made into tablets, for example, by first adding about 25 wt % to about 40 wt % of a solid pharmaceutically acceptable tablet excipient which will form a compressible mixture with the coated cores and which may be formed into a tablet without crushing the coated cores, and optionally an effective amount of a tablet disintegrating agent and a lubricant. The solid pharmaceutically acceptable tablet excipient may comprise, for example, lactose, dextrose, mannitol, calcium phosphate, microcrystalline cellulose, powdered sucrose, or combinations comprising one or more of the foregoing excipients. The tablet disintegrant may comprise crospovidone, croscarmellose sodium, dry starch, sodium starch glycolate, and the like, and combinations comprising one or more of the foregoing disintegrants. Suitable lubricants include, for example, calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants.

The pellets may be characterized by their dissolution properties. For pellet testing, a USP Type I apparatus using 500 mL 0.05 M pH 7.5 phosphate buffer as the dissolution medium at 37° C. and 50 rpm may be employed. For finished capsules, a sequential dissolution method, using a USP Type I apparatus, may be used in which the capsules are first tested in 500 mL 0.1N HCl for one hour before transferring to 500 mL pH 7.5 phosphate buffer at 100 rpm and 37° C. Dissolution may also be tested at different pHs such as, for example, pH 4.5.

For pellets, the dissolution using 500 mL 0.05 M pH 7.5 phosphate buffer as the dissolution medium is:

about 15% to about 25% release at 2 hours,

about 40% to about 60% release at 4 hours, and

about 90% to 100% release at 8 hours.

For pellets, the dissolution in 500 mL dissolution media at pH 4.5 is:

about 9% to about 16% release at 4 hours, and

about 18% to about 28% release at 8 hours.

For capsules using the capsule dissolution test, the release properties are delayed for about 1 hour compared to the pellets.

The pharmaceutical sustained release composition is provided in a unit dosage form and administration occurs at intervals of about 8 to about 24 hours. The sustained release pharmaceutical pellet composition may be administered under a similar dosage regimen to that used for Kadian®, for example. The multi-pellet encapsulated form may for example be administered every eight to twenty-four hours. The pharmaceutical pellet composition comprising a morphine compound may provide effective pain relief with once to four times daily administration. Versatility of dosing may be achieved with 10 mg, 20 mg, 50 mg, 100 mg, 200 mg, 500 mg or any other dose strength of capsules.

In accordance with a further aspect of the present invention, there is provided a method of treating pain associated conditions in patients requiring such treatment which method includes administering to a patient an effective amount of a sustained release pharmaceutical pellet composition of the present disclosure.

The present invention will now be more fully described with reference to the accompanying examples. It should be understood, however, that the following description is illustrative only and should not be taken in any way as a restriction on the generality of the invention specified above.

### COMPARATIVE EXAMPLE 1

For development of a high dose morphine dosage form, the initial strategy was to decrease the size of the sugar spheres employed for the core elements and increase the amount of morphine sulfate deposited on the cores. The coating applied for the commercially available Kadian® dosage form was employed. The core composition is given in Table 1.

TABLE 1
Core composition for examples

| Component | Percent | Weight (g) |
|---|---|---|
| Morphine sulfate | 60.08 | 8,225 |
| Sugar spheres (18-20 mesh) | 36.73 | 5,029 |
| Hypromellose | 3.19 | 439 |
| Total | 100 | 13,693 |

The cores were prepared by applying a layer of binder solution to the sugar spheres followed by applying a slurry of morphine sulfate dispersed in granulating solution in a fluid bed processor. The cores were then coated to produce pellets using the coating composition of Table 2.

TABLE 2
Coating compositions for Comparative Example 1 pellets

| Component | Percent |
|---|---|
| Ethylcellulose NF 50 cps | 5.4 |
| Polyethylene glycol 6000 | 1.9 |
| Eudragit L100-55 | 1.6 |
| Diethyl phthalate | 1.1 |
| Talc 1656 | 5.0 |
| Alcohol USP | 85.0 |
| Total | 100 |

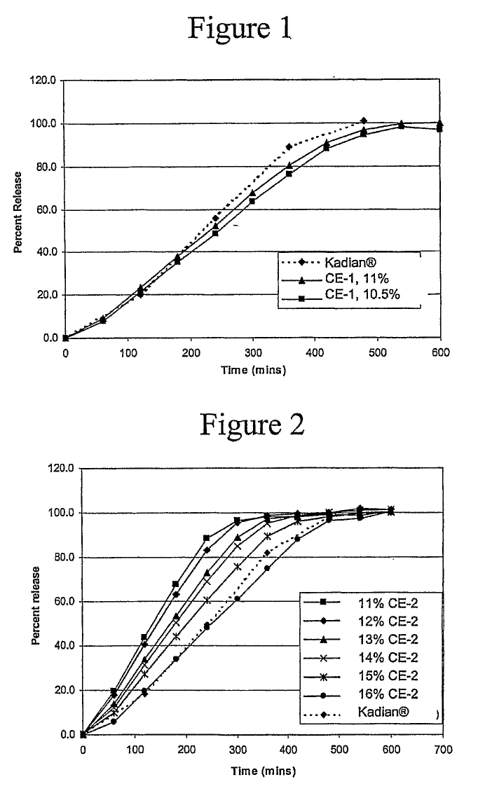

The coating composition was applied in a fluid bed apparatus. The ratio of the acid soluble polymer (polyethylene glycol) to the enteric polymer (Eudragit L100-55) was 1.2:1. The dissolution of the coated pellets (CE-1) at pH 7.5 was compared to reference Kadian® pellets as shown in FIG. 1. At coating weights of 10.5% and 11%, the high dose morphine pellets match Kadian® pellets.

### COMPARATIVE EXAMPLE 2

The coating composition was modified to increase the coat weights to about 16% to match Kadian® pellets. The composition is shown in Table 3 and the dissolution profile is shown in FIG. 2. The ratio of the acid soluble polymer (polyethylene glycol) to the enteric polymer (Eudragit L100-55) was 1.3:1.

TABLE 3
Modified coating compositions for Comparative Example 2 pellets

| Component | Percent |
|---|---|
| Ethylcellulose NF 50 cps | 5.1 |
| Polyethylene glycol 6000 | 2.1 |
| Eudragit L100-55 | 1.6 |
| Diethyl phthalate | 1.1 |
| Talc 1656 | 4.95 |
| Alcohol USP | 85.15 |
| Total | 100 |

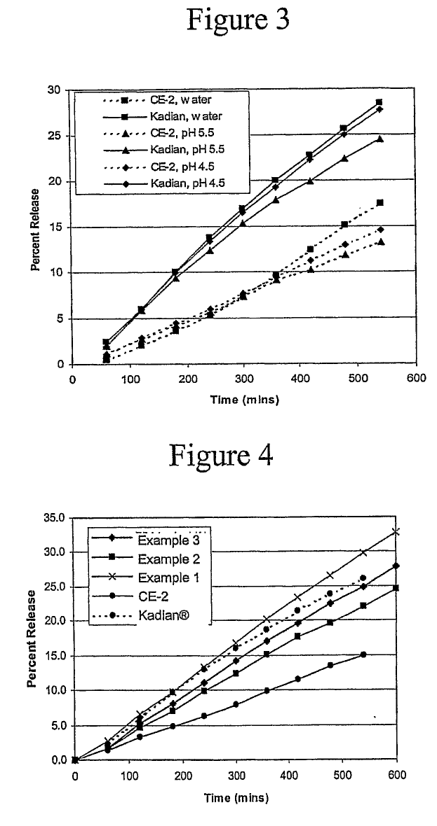

The pharmacokinetic parameters of a pellet formulation in accordance with Comparative Example 2 (CE-2) and having a 16% coating weight were compared to Kadian® pellets and did not achieve satisfactory bioequivalence. In order to determine the source of the difference in bioequivalence, the dissolution of the comparative high dose pellets was compared to Kadian® pellets at different pHs as shown in FIG. 3. As shown in FIG. 3, the comparative high dose morphine pellets have a significantly slower release profile at pHs below 6.0 which may account for the observed differences in the pharmacokinetic parameters.

In order to formulate high dose morphine cores, the percentage of sugar in the core was reduced to less than half of the commercial Kadian® pellets. Without being held to theory, it is believed that the sugar in the core of the pellets contributes as an osmotic agent and that by reducing the amount of sugar spheres in the core, the osmotic drive contributing to release is reduced. The pellet coating comprises an acid soluble polymer (polyethylene glycol) and an enteric polymer (Eudragit L100-55). At pHs above 6.0, the enteric polymer (Eudragit L100-55) is expected to dissolve and create pores to allow diffusion of the morphine from the core. The reduced osmotic drive from the sugar in the core may be compensated by faster dissolution and the pore-forming ability of the enteric polymer (Eudragit L100-55). At pHs below 6.0, however, the drug release is expected to primarily be controlled by the osmotic drive from the core because the dissolution of the enteric polymer (Eudragit L100-55) is very slow at pHs below 6.0. Thus, below pH 6.0, the reduced permeability of the coating combined with the reduced osmotic push from the core may both contribute to the reduced release rate of comparative example 1 compared to Kadian®.

### EXAMPLES 1-5

In order to increase the release of morphine from the core at pHs below 6.0, it was decided to increase the permeability of the coating at pHs below 6.0. One way to increase the coating permeability at low pH is to adjust the ratio of the acid soluble polymer (polyethylene glycol) to the enteric polymer Eudragit L100-55). Because the Eudragit L100-55 has reduced solubility below pH 5.5, it was decided to increase the proportion of polyethylene glycol to increase the permeability of the coating below pH 6.0. The morphine-coated sugar pellets were formulated similarly to what is shown in Table 1. The coating compositions are shown below in Table 4 and were coated at a thickness of 14 wt %.

TABLE 4
Coating compositions for exemplary pellets

| | Example 1, | Example 2, | Example 3, |
|---|---|---|---|
| Component | Percent | Percent | Percent |
| Ethylcellulose NF 50 cps | 5.1 | 5.1 | 5.1 |
| Polyethylene glycol 6000 | 2.3 | 2.2 | 2.25 |
| Eudragit L100-55 | 1.4 | 1.5 | 1.55 |
| Diethyl Phthalate | 1.1 | 1.1 | 1.1 |
| Talc 1565 | 4.95 | 4.95 | 4.95 |
| Alcohol USP | 85.15 | 85.15 | 85.15 |
| Total | 100 | 100 | 100 |
| Ratio polyethylene | 1.64 | 1.47 | 1.45 |
| glycol:Eudragit L100-55 | | | |

As shown in FIG. 4, the formulations in which the coating has a ratio of polyethylene glycol:Eudragit L100-55 of greater than 1.45:1 have dissolution properties at pH 4.5 that better match Kadian® pellets. Forth and fifth pellet compositions were made having a coating as described in Table 5. The coating weight was 16 wt % for Example 4 and 5.

TABLE 5
Coating compositions for exemplary pellets

| | Example 4, | Example 5, |
|---|---|---|
| Component | Percent | Percent |
| Ethylcellulose NF 50 cps | 5.0 | 5.0 |
| Polyethylene glycol 6000 | 2.3 | 2.4 |
| Eudragit L100-55 | 1.5 | 1.4 |
| Diethyl Phthalate | 1.1 | 1.1 |
| Talc 1565 | 4.95 | 4.95 |
| Alcohol USP | 85.15 | 85.15 |
| Total | 100 | 100 |
| Ratio polyethylene | 1.53 | 1.71 |
| glycol:Eudragit L100-55 | | |

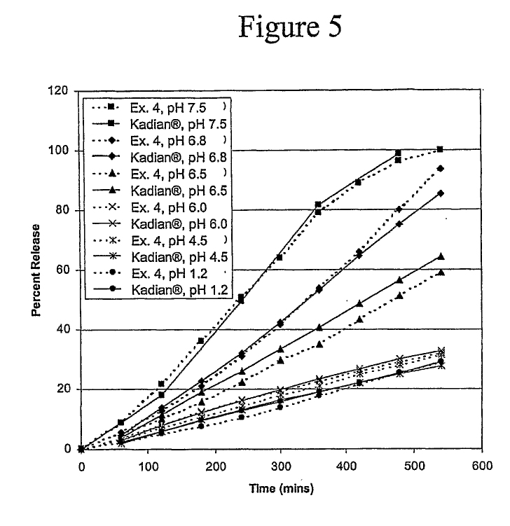

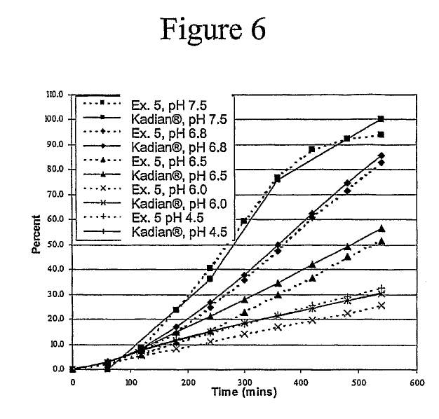

As shown in FIG. 5, the pellets of Example 4 have a similar dissolution to Kadian® pellets at pH 7.5 down to 1.2. As shown in FIG. 6, the pellets of Example 5 also have a similar dissolution to Kadian® pellets at pH 7.5 down to pH 1.2. A dosage form containing these high dose pellets are expected to be bioequivalent to Kadian®.

### EXAMPLE 6

In this example, core elements are made by a granulation/extrusion/marumerization method. Core elements are made according to the amounts shown in Table 6.

TABLE 6
Extruded core formulation

| Component | Percent | Weight (g) |
|---|---|---|
| Morphine sulfate | 85 | 2550 |
| Sugar | 11 | 330 |
| Hypromellose | 4 | 120 |
| Total | 100 | 13,693 |

The sugar, morphine sulfate, and hypromellose are granulated in a solvent and then extruded to for pellet cores. The extruded pellet cores are then coated with a coating composition according to Table 7.

TABLE 7
Coating composition for extruded cores

| | Example 6, |
|---|---|
| Component | Percent |
| Ethylcellulose NF 50 cps | 5.2 |
| Polyethylene glycol 6000 | 2.6 |
| Eudragit L100-55 | 1.1 |
| Diethyl Phthalate | 1.1 |
| Talc 1565 | 5 |
| Alcohol USP | 85 |
| Total | 100 |
| Ratio polyethylene | 2.4 |
| glycol:Eudragit L100-55 | |

### EXAMPLE 7

### Biostudy

Absorption a high dose 200 mg capsule according to the present disclosure (test, corresponds to Example 5 from above) was compared to that of an equivalent dose (2×) of the existing Kadian® 100 mg capsules (reference) under fasted and fed conditions, respectively. This was a single-dose, open-label, randomized, two-period crossover study. Data from 28 subjects who completed the study without protocol violation were included in the pharmacokinetic and statistical analyses. The results are shown in Tables 8 and 9 for fasted and fed studies respectively. Morphine-6β-Glucuronide (M6G) is a major metabolite of morphine.

TABLE 8
Statistical Analysis of the Pharmacokinetic Parameters of Morphine
Dosage Forms Under Fasted Conditions

| | | | Ratio (%) | 90% Confidence | |
|---|---|---|---|---|---|
| Pharmacokinetic | Least Squares Mean | | (Test/ | Interval* | |
| Variable | Test** | Reference** | Reference) | Lower | Upper |
| Morphine | | | | | |
| Cₘₐₓ (ng/mL) | 42.60 | 47.09 | 90.46 | 82.94 | 98.66 |
| AUCₗₐₛₜ | 783.56 | 767.59 | 102.08 | 86.85 | 105.44 |
| (ng · hr/mL) | | | | | |
| AUC_{inf} | 862.96 | 838.97 | 102.86 | 96.15 | 106.70 |
| (ng · hr/mL) | | | | | |
| M6G | | | | | |
| Cₘₐₓ (ng/mL) | 201.76 | 220.24 | 91.61 | 85.26 | 99.48 |
| AUCₗₐₛₜ | 3846.47 | 3864.39 | 99.54 | 96.69 | 103.00 |
| (ng · hr/mL) | | | | | |
| AUC_{inf} | 4109.81 | 4114.47 | 99.89 | 96.02 | 103.70 |
| (ng · hr/mL) | | | | | |

*90% Confidence Intervals are based on log-transformed data
**Test: Inventive dosage form, 200 mg administered orally under fasted conditions
Reference: Kadian 100 mg × 2 administered orally under fasted conditions

TABLE 9
Statistical Analysis of the Pharmacokinetic Parameters of
Morphine Dosage Form Under Fed Conditions

| | | | Ratio (%) | 90% Confidence | |
|---|---|---|---|---|---|
| Pharmacokinetic | Least Squares Mean | | (Test/ | Interval* | |
| Variable | Test** | Reference** | Reference) | Lower | Upper |
| Morphine | | | | | |
| Cₘₐₓ (ng/mL) | 37.34 | 32.80 | 113.84 | 95.29 | 126.50 |
| AUCₗₐₛₜ | 691.25 | 674.14 | 102.54 | 95.57 | 107.91 |
| (ng · hr/mL) | | | | | |
| AUC_{inf} | 800.25 | 789.15 | 101.41 | 95.91 | 107.88 |
| (ng · hr/mL) | | | | | |
| M6G | | | | | |
| Cₘₐₓ (ng/mL) | 221.38 | 188.92 | 117.18 | 101.96 | 132.95 |
| AUCₗₐₛₜ | 3863.73 | 3750.21 | 103.03 | 99.54 | 106.82 |
| (ng · hr/mL) | | | | | |
| AUC_{inf} | 4294.65 | 4247.84 | 101.10 | 96.91 | 105.43 |
| (ng · hr/mL) | | | | | |

*90% Confidence Interval are based on log-transformed data
**Test: Inventive dosage form, 200 mg administered orally under fasted conditions
Reference: Kadian 100 mg × 2 administered orally under fasted conditions

The test 200 mg capsules are bioequivalent to an equivalent dose given as the existing Kadian® 100 mg capsules under fasted conditions.

Under fed conditions, the extent of absorption of the test 200 mg capsules (AUC) is substantially equivalent to an equivalent dose given as the existing Kadian® 100 mg capsules. However, with respect to Cₘₐₓ, the inventive 200 mg capsules are about 14% to about 17% higher than the existing Kadian® 100 mg capsules at an equivalent dose when administered under fed conditions. The test dosage form, with respect to Cₘₐₓ, have about at 12% difference between dosing under the fed state versus the fasted state, while the reference Kadian® pellets have about a 32% difference between dosing under the fed state versus the fasted state. Thus, the inventive dosage form has a smaller difference between dosing in the fed state versus the fasted state than Kadian®.

All ranges disclosed herein are inclusive and combinable. Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A dosage form comprising a plurality of pellets, the pellets comprising:
a core element comprising morphine sulfate, a filler and a binder, wherein the morphine compound, calculated as the anhydrous form, comprises about 50 wt % to about 85 wt % of the total weight of the core element; and
a controlled-release coating disposed on at least a portion of the core element, the coating comprising an insoluble matrix polymer which is insoluble at pH 1 to 7.5; an enteric polymer which is insoluble at pH 1 to 4 and soluble at pH 6 to 7.5; and an acid soluble polymer which is soluble at a pH of 1 to 4, wherein the ratio of the acid soluble polymer to the enteric polymer is 1.45:1 to 2.5:1 on a weight basis,
wherein the Cₘₐₓ of the dosage form differs by less than 20% when administered to a mammalian subject in the fed state compared to the fasted state.

2. The dosage form of claim 1, wherein the AUC_{0-∞} of the dosage form differs by less than 20% when administered to a mammalian subject in the fed state compared to the fasted state.

3. The dosage form of claim 1, wherein the morphine sulfate comprises 60 wt % to about 80 wt % of the core element.

4. The dosage form of claim 1, wherein the Cₘₐₓ of the dosage form differs by less than 15% when administered to a mammalian subject in the fed state compared to the fasted state.

5. The dosage form of claim 1, wherein the AUC_{0-∞} of the dosage form differs by less than 15% when administered to a mammalian subject in the fed state compared to the fasted state.

6. The dosage form of claim 1, wherein the ratio of the acid soluble polymer to the enteric polymer is 1.5:1 to 2:1.

7. The dosage form of claim 1, wherein the insoluble matrix polymer comprises about 1 wt % to about 85 wt %, the acid soluble polymer comprises about 25 wt % to about 60 wt % of the coating, and the enteric polymer comprises about 1 wt % to about 24 wt % of the total weight of the coating.

8. The dosage form of claim 1, wherein the controlled-release coating comprises about 8 wt % to about 17 wt % of the total weight of the pellet composition.

9. The dosage form of claim 1, wherein the acid soluble polymer is polyvinyl pyrrolidone, hydroxypropyl cellulose, hydroxypropyl methylcellulose, polyethylene glycol having a molecular weight of 1700 to 20,000, polyvinyl alcohol, or a combination comprising one or more of the foregoing polymers.

10. The dosage form of claim 1, wherein the enteric polymer is cellulose acetate phthalate, hydroxypropyl methylcellulose phthalate, polyvinyl acetate phthalate, a methacrylic acid copolymer, hydroxypropyl methylcellulose acetate succinate, shellac, cellulose acetate trimellitate, or a combination comprising one or more of the foregoing enteric polymers.

11. The dosage form of claim 1, wherein the filler comprises an inert core.

12. The dosage form of claim 11, wherein the inert core comprises a sugar sphere.

13. The dosage form of claim 1, comprising 100 mg of morphine sulfate.

14. The dosage form of claim 1, comprising 200 mg of morphine sulfate.

15. A method of increasing patient compliance, comprising administering to a patient in need thereof a dosage form comprising a plurality of pellets, the pellets comprising:
a core element comprising morphine sulfate, a filler and a binder, wherein the morphine sulfate, calculated as the anhydrous form, comprises about 50 wt % to about 85 wt % of the total weight of the core element; and
a controlled-release coating disposed on at least a portion of the core element, the coating comprising an insoluble matrix polymer which is insoluble at pH 1 to 7.5; an enteric polymer which is insoluble at pH 1 to 4 and soluble at pH 6 to 7.5; and an acid soluble polymer which is soluble at a pH of 1 to 4, wherein the ratio of the acid soluble polymer to the enteric polymer is 1.45:1 to 2.5:1 on a weight basis,
wherein the Cₘₐₓ of the dosage form differs by less than 20% when administered to a mammalian subject in the fed state compared to the fasted state.

16. The method of claim 15, wherein the AUC_{0-∞} of the dosage form differs by less than 20% when administered to a mammalian subject in the fed state compared to the fasted state.

17. The method of claim 15, wherein the morphine sulfate comprises 60 wt % to about 80 wt % of the core element.

18. The method form of claim 15, wherein the Cₘₐₓ of the dosage form differs by less than 15% when administered to a mammalian subject in the fed state compared to the fasted state.

19. The method of claim 15, wherein the AUC_{0-∞} of the dosage form differs by less than 15% when administered to a mammalian subject in the fed state compared to the fasted state.

20. The method of claim 15, wherein the ratio of the acid soluble polymer to the enteric polymer is 1.5:1 to 2:1.
